(19) United States
(12) Patent Application Publication
(10) Pub. No.: US 2013/0136668 A1
(43) Pub. Date: May 30, 2013

(54) MACHINE FOR STERILIZING THE TOPSOIL OF THE GROUND

(71) Applicant: Robert Hernandez, Perpignan (FR)

(72) Inventor: Robert Hernandez, Perpignan (FR)

(73) Assignee: INNOVAPEUR, MONTESCOT (FR)

(21) Appl. No.: 13/814,802

(22) PCT Filed: Aug. 11, 2011
(86) PCT No.: PCT/FR11/51904

§ 371 Date: Feb. 7, 2013

(30) Foreign Application Priority Data
Aug. 17, 2010 (FR) 10/03386

(51) Int. Cl. A61L 2/20

(52) U.S. Cl. CPC A61L 2/20
USPC 422/292

(57) ABSTRACT

A machine for sterilizing the topsoil of the ground, including the injection of superheated steam, includes a water tank (5) and elements for producing superheated steam and for conveying the latter to at least one supply nozzle (6). The elements for producing superheated steam include: a spraying unit (8) that is in communication with the water tank and that includes a spraying chamber (9) in which a spraying device (10) is disposed in order to form an aerosol of fine water particles in the chamber; a unit (11) which can generate a conveying airflow that is moved through the spraying chamber (9) so as to drive the prepared aerosol out of the chamber; and a heating unit (12) which can raise the temperature of the aerosol such that the latter is converted into steam.

### TECHNICAL FIELD

This invention is of the field of equipment used in agriculture for the purpose of sterilizing topsoil using superheated steam, and it relates more particularly to a machine for spraying steam jets onto and into the topsoil.

### STATE OF THE PRIOR ART

For the purpose of removing from topsoil the vermin that may infest it, it is known to spread phytosanitary products on and in the ground before and after the sowing. Although such a technique offers the advantage of being quick to implement, it is, however, responsible for severe and persistent pollution of the environment. Moreover, the products require particular precautions and constraints; in particular the personnel responsible for spreading have to be properly protected by appropriate suits, and after spreading, the equipment that is used should be carefully cleaned. Furthermore, the cleaning agent used, typically water, is to be collected and stored in dedicated tanks for the purpose of subsequent clean-up treatment, which is costly in itself.

Treatments are also known that do not consist in spreading phytosanitary products but rather in spraying the superheated water vapor into the ground so as to use the sterilizing power of heat.

Such techniques offer the advantage of not generating any pollution, but the machines that use these techniques have the drawback of being relatively costly to implement and use. This essentially resides in the fact that the steam production sources with which these machines are equipped have a relatively modest output and use, in order to ensure the production of superheated steam, a distiller that uses the combustion of fossil fuel, typically diesel fuel, of increasingly high cost.

In addition, with such machines, a significant consumption of water was observed, which involves the presence of a high-capacity tank inducing a noteworthy increase in weight.

Finally, the output from these machines, in terms of surface area treated per unit of time, still exceeds that which can be obtained by phytosanitary treatments, and thus this last method, despite all of its drawbacks, is still widely used today.

In the past, an attempt was made to solve the problems of the output of the steam treatment machines. Thus, a self-propelled machine is known whose power plant consists of an internal combustion engine. The exhaust gases are collected and used for sterilizing the soil. The drawback of this solution is the introduction of polluting elements into the topsoil.

From patent FR 2 902 285, a machine is known that uses an air compressor staged at a high compression level that is suitable for delivering a superheated air stream that can, on the one hand, ensure the vaporization of the water, and, on the other hand, convey the formed water vapor, toward supply nozzles as a steam production source. The primary advantage of this arrangement is to eliminate the need for the use of distillers and to prevent the introduction of polluting elements into the ground, but such a machine conveys an additional weight constituted by the air compressor in addition to the engine.

A piece of equipment for farm use for the injection of a pressurized fluid into the ground and a vehicle comprising such a piece of equipment are also known from the patent application WO2009021877.

Essentially, the piece of equipment that is installed at the rear of the vehicle consists of a horizontal drum that is driven in rotation along a horizontal axis that is transverse to the direction of advance of the vehicle. This hollow drum is connected by a rotary joint to a steam production unit and comprises a series of hollow radial arms carrying steam injectors. During the rotation of the drum, the radial arms are secured to penetrate into the ground to inject steam therein and to participate in the propulsive force.

Such a vehicle does not perform a function of loosening the soil, enabling the diffusion of steam into the ground. In addition, to the extent that the propulsive force of the vehicle is partly produced by the radial arms of the drum, the latter can be entrained only in a so-called “swallowing” direction.

Machines for treating soil using superheated water vapor are also known from the patent application EP 1 479 287 and the international application WO0207502. These machines use teeth carrying hot water vapor injection nozzles connected to a power plant for steam generation. These teeth are provided for penetrating into the ground so as to inject steam therein.

### DISCLOSURE OF THE INVENTION

This invention has as its object to resolve the previously-cited drawbacks by using a self-propelled machine for treatment of soil by water vapor, with an improved output.

For this purpose, the self-propelled machine for soil treatment by steam according to the invention—comprising a propulsion set equipped with an internal combustion engine, a water tank, and means for producing at least one stream of superheated water vapor brought to one or more supply nozzles—is essentially characterized in that the means for production of superheated steam comprise:
A spraying unit in communication with the water tank, with said unit comprising a spraying chamber in which a spraying device is placed to form an aerosol that consists of fine water particles,
A unit that is capable of generating a stream of conveying air that is run through said spraying chamber to entrain the formed aerosol outside of this chamber,
A heating unit to raise the temperature of the aerosol in such a way that the latter is transformed into steam.

The division into fine droplets of water makes it possible to facilitate the subsequent vaporization and considerably improves the output of the machine in terms of value of the surface treated per unit of time. In addition, the division into fine droplets of the water makes possible an optimal vaporization, which is a factor for reducing water consumption.

According to another characteristic of the invention, the spraying chamber is formed in the tank, above the maximum water level, and the stream of carrier air passes through the upper part of the tank, with this upper part forming a mixing chamber.

Preferably, according to another characteristic of the invention, the spraying device comprises at least one ultrasonic head mounted in the upper part of the water tank above the maximum liquid level.

Advantageously, according to another characteristic of the invention, the device for heating the air stream comprises an air/air heat exchanger equipped with a first path through which exhaust gases from the internal combustion engine pass and a second path through which the mixture of carrier air stream and the aerosol passes, with this second path being in heat exchange with the first path through at least one heat exchange wall and in airtight communication with the spraying chamber.

The advantage of such an arrangement is to recover the heat energy of the exhaust gases to heat the air stream and aerosol mixture so as to vaporize water while preventing the pollution of the topsoil by these exhaust gases.

According to another characteristic of the invention, the heat exchanger is arranged downstream from the spraying chamber, in which case the second path is also in airtight communication with the spraying nozzle(s).

As a variant, according to still another characteristic of the invention, the heat exchanger is arranged upstream. In this configuration, the carried air is brought to a high temperature before penetrating into the spraying chamber.

According to another characteristic of the invention, the machine is equipped with a device for loosening the topsoil, with this device being equipped with a support structure that carries at least one earth-working tooth equipped in its active part (part provided for coming into the ground and loosening it) with at least one nozzle for supply of the hot air stream loaded with superheated water vapor.

Such an arrangement makes it possible, while loosening the topsoil, to introduce the superheated water vapor into the thickness of the latter, which greatly improves the effectiveness of the treatment.

According to another characteristic of the invention, the supply nozzle or each supply nozzle is in communication with an inner channel made in the earth-working tooth, with this inner channel being in communication with a second channel made in the support structure of the tooth, this second channel being in communication via a feed pipe with the outlet of the second path of the last heat exchanger.

According to another characteristic of the invention, the tooth support structure is a rotor driven by a movement transmission coupled to the output shaft of the internal combustion engine, a rotary joint being placed between the second channel and the feed pipe, with said rotor carrying at least one tooth whose inner channel is in communication with the second channel made in the support structure, with this second channel being axially formed in the rotor that constitutes this structure.

According to another characteristic of the invention, the machine comprises a second device for loosening the earth, the latter occupying an upstream position relative to the first.

### SUMMARY DESCRIPTION OF THE FIGURES OF THE DRAWINGS

Other advantages, objects and characteristics of the invention will emerge from reading the description of a preferred embodiment provided by way of a non-limiting example and referring to the accompanying drawings, in which:

FIG. 1 is a diagrammatic view of a machine according to the invention,

FIG. 2 is a profile view in diagrammatic form of a machine according to the invention,

FIG. 3 is a perspective view showing a system for lifting the working rotor from the earth,

FIG. 4 is a cutaway view showing a detail of the lifting mechanism.

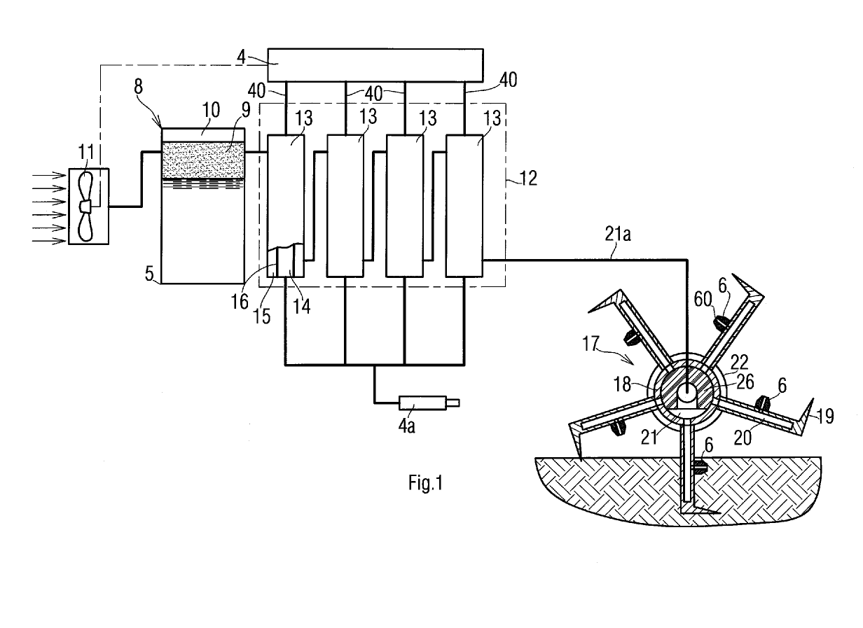

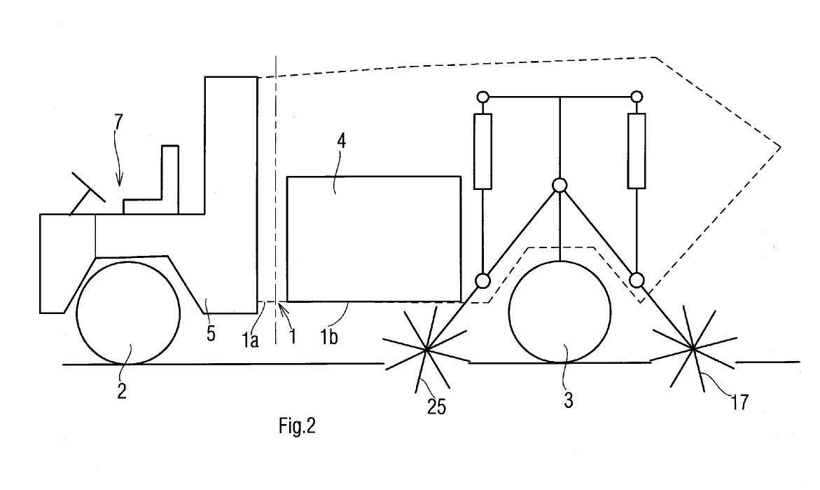

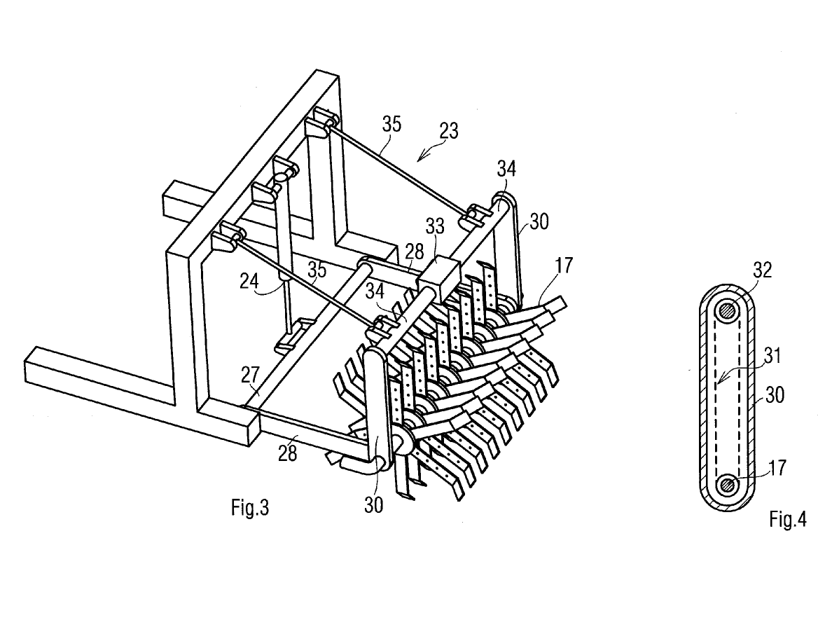

### BETTER WAY TO IMPLEMENT THE INVENTION

As shown, the self-propelled machine according to the invention comprises a frame 1 that is mounted on rolling elements arranged in a guide train 2 and a drive train 3, with said frame 1 carrying in particular a propulsion set 4 preferably formed by an internal combustion engine equipped with a rotary output shaft engaged with a movement transmission coupled to the drive train 3 of the machine.

Furthermore, the frame 1 is equipped with a water tank 5, means for producing superheated water vapor in communication, on the one hand, with the tank 5, and, on the other hand, with a nozzle 6 for supply of water vapor in the topsoil of the ground to be sterilized, with this nozzle 6 having a through inner channel 60 provided with an injector in the ground for the water vapor stream to pass through.

Preferably, the frame 1 comprises, on the one hand, a front part 1a equipped with the guide train 2, the water tank 5, and a control station 7, and, on the other hand, a rear part 1b articulated with the preceding part along a vertical axis, with this rear part 1b being equipped in particular with the drive train 3 and the propulsion set 4.

According to the preferred embodiment, the means for producing water vapor comprise:
A spraying unit 8 in communication with the water tank 5, with said unit comprising a spraying chamber 9 in which a spraying device 10 is arranged to form therein an aerosol that consists of fine water particles,
A unit 11 that can generate a stream of conveying air brought in to pass through the spraying chamber 9 to entrain, outside of this chamber, the aerosol that is formed,
A heating unit 12 that is capable of raising the temperature of the aerosol in such a way that the latter is converted into steam.

The spraying unit 8 is preferably arranged in the tank 5 above the maximum water level, and the spraying chamber 9 is formed in the tank above the maximum water level.

The spraying device 10 is attached to the walls of the tank 5 and preferably consists of one or more heads that are capable of emitting ultrasound under the action of which the surface layer of water in the tank is sprayed in fine droplets so as to form an aerosol.

The unit 11 that is capable of generating the stream of conveying air essentially consists of a blower that integrates, in a suitable shell, a fan driven by the internal combustion engine of the propulsion set 4 by means of a movement transmission. This blower is in communication via a suitable pipe with the volume of the spraying chamber 9. More particularly, this pipe is connected to an air intake opening formed in the wall of said chamber. Opposite this air intake opening, the spraying chamber 9 comprises an outlet opening by means of which it is in communication with the supply nozzles 6. It is therefore understood that the air stream is brought in to pass through the spraying chamber 9 and to entrain the aerosol that is formed by the spraying means outside of this chamber during its passage.

In the preferred embodiment, the heating unit 12 is arranged downstream from the spraying chamber 9 and is in communication, on the one hand, with said chamber 9, and, on the other hand, with the supply nozzles 6, and more particularly with the inner channel 60 of each of the latter. The stream of aerosol-loaded air passes through this heating unit 12, whose purpose is to yield to the formed mixture the calories that are necessary to the vaporization of water droplets comprising the aerosol.

In the preferred embodiment, the heating unit 12 comprises at least one heat exchanger 13 of the air/air type that is equipped with a first path 14 through which exhaust gases from at least one working chamber of the internal combustion engine pass and a second path 15 through which the stream of carrier air and the aerosol pass. This second path 15 is in heat exchange with the first path 14 through at least one heat exchange wall 16 and is in airtight communication as much with the spraying chamber 9 as with the supply nozzles 6.

Preferably, the machine comprises as many heat exchangers 13 as the internal combustion engine has working chambers, with the exhaust pipe of each working chamber being connected by a suitable pipe 40 to the first path 14 of the associated heat exchanger. Thus, each working chamber supplies one heat exchanger 13 and one only, and the outlet of the first path 14 of each heat exchanger 13 is connected to a common exhaust pot. The heat exchangers are connected in series by their second paths 15. In this way, the stream of conveying air that is loaded with sprayed water and with water vapor passes successively through the different heat exchangers 13, which gradually increases the temperature of this mixture and brings it above the water vaporization temperature.

Thus, at the outlet of the heat exchangers 13, a stream of superheated air that is loaded with superheated water vapor will be obtained. By way of purely indicative example, the temperature that is obtained at the outlet of the heat exchangers is on the order of 280 degrees Celsius.

The outlet of the second path 15 of the last heat exchanger is in communication through a pipe 21a with the supply nozzles 6.

In the preferred embodiment, the supply nozzles 6 are carried by a device 17 for loosening the topsoil of the ground. This device 17 consists of a support structure 18 that carries several earth-working teeth 19, each equipped, in their active parts, with at least one supply nozzle 6.

In the preferred embodiment, each tooth 19 comprises an inner channel 20, and the supply nozzle 6 or each supply nozzle 6, carried by this tooth, is in communication with the channel 20 via its through inner channel 61. This inner channel 20 is in communication with a second channel 21 made in the support structure 18 of the tooth 19, with this second channel 21 being in communication with the outlet of the second path of the last heat exchanger by means of a feed pipe 21a.

Preferably, the tooth support 18 is a rotor driven by a movement transmission coupled to the output shaft of the internal combustion engine. The channel 21 is formed in the rotor along the longitudinal axis of the latter and comes in the form of a bore. A rotary joint 22 is arranged between the second channel 21 and the feed pipe.

According to a preferred embodiment, the loosening device 17 is mounted on an apparatus 23 that can move between a high position and/or a rest position along which the loosening device is arranged above and separated from the ground, and a low position or active position according to which this loosening device penetrates the soil to loosen it. The movable apparatus 23 will be combined with a lifting drive element 24, of the pneumatic or hydraulic jack type, for example. By the command of this drive element, the movable apparatus 23 will either be lowered or lifted.

The earth-working rotor 17 can have a horizontal axis of rotation, and in this case, the rotary joint 22 will be combined with a distributor 26 that is known in the art, capable of supplying each tooth with hot air and steam during its travel in the earth and of interrupting this supply when the tooth is above the ground. Because of this arrangement, any waste losses of steam will be prevented.

The movable apparatus 23 consists of a pivoting structure formed by a horizontal shaft 27 that is engaged in rotation in two end bearings integral with the frame, with said shaft 27 carrying two parallel, side radial arms 28, each equipped at some distance from the shaft with a support bearing in which the rotor 17 is engaged. The horizontal shaft 27 is equipped with a radial lever arm 29 to which is articulated the end of the rod of the lifting jack 24 that is articulated by its body to the frame of the machine. Two hollow rigid flanges 30—each capable of accommodating a movement transmission 31 engaged with a transmission half-shaft 32 driven by a drive element 33 carried by a brace bar 34 rigidly joining the two hollow flanges to one another, with this brace bar 34 being hollow to accommodate the transmission half-shafts 32—are articulated to the radial arms 28, coaxially to the rotor 17. This bar 34 accommodates or the flanges 30 accommodate in articulation two connecting rods 35 that are further articulated to the frame of the machine. The connecting rods 35, the flanges 30, the radial arms 28 and the frame form a deformable parallelogram structure.

Each transmission 31 of movement between the corresponding half-shaft 32 and the rotor 17 will consist of two gear wheels that are respectively wedged on the half-shaft and on the rotor and by an articulated-mesh chain engaged with the two cogs.

As a variant, the device for loosening the earth consists of at least one rotary harrow with a vertical axis of rotation. This rotary harrow comprises a rotor with a vertical axis of rotation to which the earth-working teeth 19 are attached. According to this arrangement, the teeth 19 of the rotor simultaneously work the soil, and the steam is distributed simultaneously to various nozzles of the different teeth. Thus, the rotor does not have a distributor.

Advantageously, in front of the loosening device 17, the machine according to the invention comprises a second loosening device 25 in the form of a rotor with teeth. This device has as its object to carry out a preparatory loosening so as to subsequently promote the diffusion of steam in the topsoil of the ground.

1. Machine for sterilizing topsoil before sowing, by injection of superheated water vapor in the topsoil, comprising a frame (1) that is mounted on rolling elements arranged in trains and on which frame are installed a propulsion set (4) that is capable of delivering a drive torque communicated to at least one of the wheel trains, a water tank (5), and means for producing superheated water vapor and for conveying the latter to at least one supply nozzle (6), characterized in that the means for producing superheated water vapor comprise:
A spraying unit (8) in communication with the water tank, whereby said unit comprises a spraying chamber (9) in which a spraying device (10) is placed to form an aerosol that consists of fine water particles,
A unit (11) that is capable of generating a stream of conveying air that is run through said spraying chamber (9) to entrain the formed aerosol outside of this chamber,
A heating unit (12) for raising the temperature of the aerosol in such a way that the latter is transformed into steam.

2. Machine according to claim 1, wherein the spraying chamber (9) is formed in the tank (5) above the maximum water level.

3. Machine according to claim 2, wherein the spraying device (9) comprises at least one ultrasonic head.

4. Machine according to claim 1, wherein the propulsion set comprises an internal combustion engine and the heating unit (12) comprises at least one air/air heat exchanger (13) that is equipped with a first path (14) through which the exhaust gases from at least one working chamber of the internal combustion engine pass and a second path (15) through which the carrier air stream passes, with this second path being in heat exchange with the first path through at least one heat exchange wall (16) and in airtight communication with the spraying chamber (9).

5. Machine, according to claim 4, wherein the heat exchanger (13) is installed downstream from the spraying chamber (9) and through which the mixture of carrier air stream and aerosol passes, with the second path (15) of the heat exchanger (13) being in communication with the supply nozzles (6).

6. Machine according to claim 5, wherein it comprises as many heat exchangers (13) as the internal combustion engine has working chambers, wherein each working chamber supplies one heat exchanger (13) and one only that is specific thereto, wherein the exhaust pipe of each working chamber is connected by a suitable pipe (40) to the first path (14) of the associated heat exchanger, and wherein the heat exchangers (13) are connected in series by their second paths (15).

7. Machine according to claim 1, wherein it is equipped with a device (17) for loosening topsoil, with this device (17) being equipped with a support structure (18) that carries at least one earth-working tooth (19) equipped in its active part with at least one supply nozzle (6).

8. Machine according to claim 7, wherein the supply nozzle (6) or each supply nozzle (6) is in communication with an inner channel (20) made in the earth-working tooth (19), with this inner channel (20) being in communication with a second channel (21) made in the support structure of the tooth, this second channel (21) being in communication via a feed pipe (21a) with the outlet of the second path (15) of the last heat exchanger (13).

9. Machine according to claim 8, wherein the tooth support structure (18) is a rotor driven by a movement transmission coupled to the output shaft of the internal combustion engine, a rotary joint (22) being placed between the second channel and the feed pipe, with said rotor carrying at least one tooth (19) whose inner channel (20) is in communication with the second channel (21) made in the support structure, with this second channel (21) being axially formed in the rotor that constitutes this structure.

10. Machine according to claim 9, wherein the rotor (18) has a horizontal axis of rotation.

11. Machine according to claim 8, wherein the tooth support structure (18) is a rotary harrow with a vertical axis of rotation.

12. Machine according to claim 7, wherein it comprises a second loosening device (25), mounted in front of the first device.

13. Machine according to claim 8, wherein it comprises a second loosening device (25), mounted in front of the first device.

14. Machine according to claim 9, wherein it comprises a second loosening device (25), mounted in front of the first device.

15. Machine according to claim 10, wherein it comprises a second loosening device (25), mounted in front of the first device.

16. Machine according to claim 11, wherein it comprises a second loosening device (25), mounted in front of the first device.
